(19) United States
(12) Plant Patent Application Publication
(10) Pub. No.: US 2002/0148011 P1
(43) Pub. Date: Oct. 10, 2002

(54) MINIATURE ROSE VARIETY 'POULgo004'

(75) Inventors: L. Pernille Olesen, Fredensborg (DK); Mogens N. Olesen, Fredensborg (DK)

Correspondence Address: Mogens N. Olesen,, Poulsen Roser Pacific, Inc., 2550 Old Military Road, Central Point, OR, 97502, US

(73) Assignee: Poulsen Roser Aps.

(21) Appl. No.: 09/776,154

(22) Filed: Feb. 2, 2001

(51) Int. Cl. A01H 5/00

(52) U.S. Cl. PLT/121

(57) ABSTRACT

A new miniature rose plant which has abundant, light red flowers and attractive foliage. This new and distinct variety has shown to be uniform and stable in the resulting generations from asexual propagation.

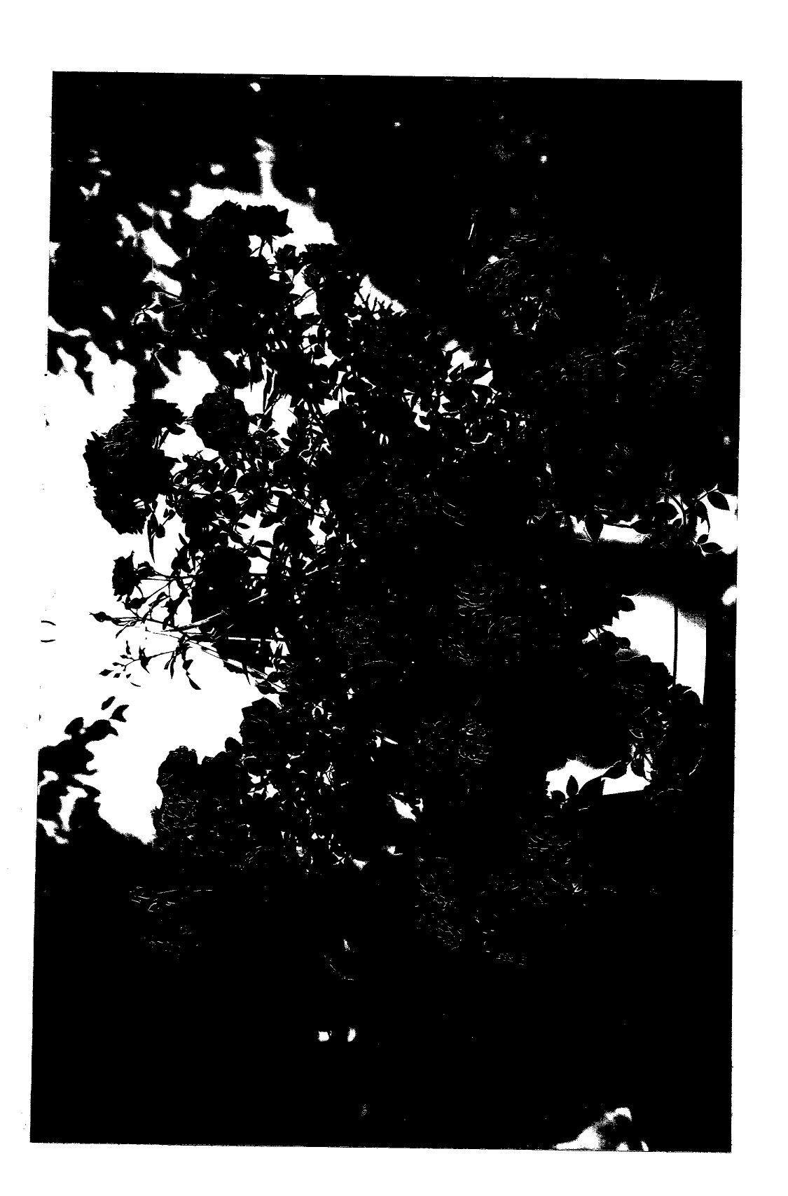

### SUMMARY OF THE INVENTION

The present invention constitutes a new and distinct variety of miniature rose plant which originated from a controlled crossing between an unnamed, unpatented seedling and ‘POULrijk’, described and illustrated under U.S. Plant patent application Ser. No. 09/261,445, dated Mar. 3, 1999. The two parents were crossed during the summer of 1994 and the resulting seeds were planted in a controlled environment in Fredensborg, Denmark. The new variety is named ‘POULgo004’.

The new rose may be distinguished from its seed parent, an unnamed seedling, by the following combination of characteristics:

1. The seed parent exhibits pink blooms, where ‘POULgo004’ exhibits red blooms.

The new variety may be distinguished from its pollen parent, ‘POULrijk’ by the following combination of characteristics:

1. ‘POULrijk’ exhibits pink blooms, where those of ‘POULgo004’ are red.

2. ‘POULrijk’ is a shrub rose with a spreading, low-growing habit, while ‘POULgo004’ is more procumbent and suitable for use in hanging baskets.

The objective of the hybridization of this rose variety was to create a new and distinct variety for use in hanging baskets with unique qualities, such as:

1. Uniform and abundant flowers;

2. Vigorous, but compact growth when propagated by cuttings;

3. Disease resistance;

4. A miniature rose suitable for use in hanging basket containers in the nursery industry.

This combination of qualities is not present in previously available commercial cultivars of this type and distinguish ‘POULgo004’ from all other varieties of which we are aware.

As part of their rose development program, L. Pernille Olesen and Mogens N. Olesen germinated the seeds from the aforementioned hybridization during winter 1994 and conducted evaluations on the resulting seedlings in a controlled environment in Fredensborg, Denmark.

‘POULgo004’ was selected in the spring 1995 by the inventors as a single plant from the progeny of the aforementioned hybridization.

Asexual reproduction of ‘POULgo004’ by traditional budding and rooted cuttings was first done by L. Pernille and Mogens N. Olesen in their nursery in Fredensborg, Denmark in July, 1995. This initial and other subsequent asexual propagations conducted in controlled environments have demonstrated that the characteristics of ‘POULgo004’ are true to type and are transmitted from one generation to the next.

### BRIEF DESCRIPTION OF THE DRAWING

The accompanying color illustration shows as true as is reasonably possible to obtain in color photographs of this type the variety rose ‘POULgo004’. Specifically illustrated in SHEET 1 is a plant of ‘POULgo004’ growing in a 20-cm hanging basket.

### DETAILED DESCRIPTION OF THE VARIETY

The following is a description of ‘POULgo004’, as observed in its growth during trials at the testing station Prüfstelle Rethmar under the te sting authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULrijk’, a rose variety from the same inventors described and illustrated in U.S. Plant patent application Ser. No. 09/261,445, filed Mar. 3, 1999 are compared to ‘POULgo004’ in Chart 1.

CHART 1

| | ‘POULgo004’ | ‘POULrijk’ |
|---|---|---|
| Color of open | Red Group 53D to | Red-Purple Group |
| flower, outer | Red-Purple Group | 62B. |
| side, middle | 57A | |
| zone. | | |
| Petalage | Single | Single |
| Upon opening, | Red Group 53C | Green-White Group |
| color basal | | 157D. |
| petal spot, | | |
| outer side | | |

Parents:

Seed parent.—An unnamed seedling.

Pollen parent.—‘POULrijk’.

Classification:

Botanical.—Rosa hybrida.

Commercial.—Miniature.

### FLOWER AND FLOWER BUD

Blooming habit: Continuous.

Flower bud:

Bud form.—Ovate.

Sepals.—Weak appendages.

Receptacle.—Size: Small

Peduncle.—Surface: Few hairs and prickles exhibited.

Borne.—Many flowering shoots per stem.

Flower bloom:

Fragrance.—Weak.

Size.—Very small.

Form.—Shape of flower when viewed from the side: Upper part: Flat. Lower part: Flattened convex. Viewed from above: Irregularly rounded.

Petalage.—Single.

Color:

Petals.—Inner Side: Middle zone: A blend of Red Group 53D and Red-Purple Group 57A. Marginal zone: A blend of Red Group 53D and Red-Purple Group 57A. Basal petal spot: Yellow Group 4D. Outer side: Middle zone: A blend of Red Group 53D and Red-Purple Group 57A. Marginal zone: A blend of Red Group 53D and Red-Purple Group 57A. Basal petal spot: Yellow Group 4D.

General Tonality: A blend of Red Group 53D and Red-Purple Group 57A. No change in the general tonality at the end of the 10th day.

Petals:

Petal reflex.—Somewhat reflexed.

Undulation of margin.—Weak to medium.

Size.—Small.

Reproductive Organs:

Filaments.—Color: Yellow

Hips.—Shape: Pitcher-shaped.

### PLANT

Plant growth: Spreading. Procumbent to arching habit. Height: 40 to 60 cm.

Stems:

Thorns.—Incidence: Few to medium. Size: Both small and medium sized prickles. Shape: Concave.

Plant foliage:

Leaf size.—Small.

Glossiness.—Weak.

Color.—Upper Leaf Surface: Dark green Juvenile foliage: Exhibits anthocyanin on young shoots. Anthocyanin intonation: Color: Reddish brown.

Plant leaflets:

Leaflet.—Cross Section: Slightly concave Margin Undulation: Very weak. Other: Matte surface.

Terminal leaflet.—Length of blade: Short. Width of blade: Narrow. Shape of base: Rounded.

Disease resistance: Above average resistance to mildew, rust, black spot, and Botrytis under normal growing conditions in Hannover, Germany and Fredensborg, Denmark.

Cold Hardiness: The variety ‘POULgo004’ has been found to be cold hardy in Fredensborg, Denmark; and Hannover, Germany.

1. A new and distinct variety of rose plant of the miniature rose class, substantially as herein illustrated and described as a distinct and novel rose variety due to its abundant red flowers, procumbent to arching habit, disease resistance, and extended period of bloom.
